SEQUENCE LISTING (108 sequences)

SEQ ID NO: 1 - protein, 14 aa, Artificial Sequence
  Peptide Marker for ACHD_383_395
YISKAEEYFLLKSG

SEQ ID NO: 2 - protein, 14 aa, Artificial Sequence
  Peptide Marker for AMPE_5_17
EREGSKRYCIQTKG

SEQ ID NO: 3 - protein, 14 aa, Artificial Sequence
  Peptide Marker for ANXA1_14_26
IENEEQEYVQTVKG

SEQ ID NO: 4 - protein, 14 aa, Artificial Sequence
  Peptide Marker for ANXA2_17_29
HSTPPSAYGSVKAG

SEQ ID NO: 5 - protein, 14 aa, Artificial Sequence
  Peptide Marker for B3AT_39_51
TEATATDYHTTSHG

SEQ ID NO: 6 - protein, 14 aa, Artificial Sequence
  Peptide Marker for CALM_93_105
FDKDGNGYISAAEG

SEQ ID NO: 7 - protein, 14 aa, Artificial Sequence
  Peptide Marker for CALM_95_107
KDGNGYISAAELRG

SEQ ID NO: 8 - protein, 14 aa, Artificial Sequence
  Peptide Marker for CBL_693_705
EGEEDTEYMTPSSG

SEQ ID NO: 9 - protein, 14 aa, Artificial Sequence
  Peptide Marker for CD3Z_116_128
KDKMAEAYSEIGMG

SEQ ID NO: 10 - protein, 14 aa, Artificial Sequence
  Peptide Marker for CD79A_181_193
EYEDENLYEGLNLG

SEQ ID NO: 11 - protein, 14 aa, Artificial Sequence
  Peptide Marker for CDK2_8_20
EKIGEGTYGVVYKG

SEQ ID NO: 12 - protein, 16 aa, Artificial Sequence
  Peptide Marker for CDK3_12_26
EGTYGVVYKAKNRETG

SEQ ID NO: 13 - protein, 13 aa, Artificial Sequence
  Peptide Marker for CTNB1_79_91
VADIDGQYAMTRA

SEQ ID NO: 14 - protein, 14 aa, Artificial Sequence
  Peptide Marker for DCX_109_121
GIVYAVSSDRFRSG

SEQ ID NO: 15 - protein, 14 aa, Artificial Sequence
  Peptide Marker for DDR1_506_518
LLLSNPAYRLLLAG

SEQ ID NO: 16 - protein, 14 aa, Artificial Sequence
  Peptide Marker for DYR1A_212_224
KHDTEMKYYIVHLG

SEQ ID NO: 17 - protein, 14 aa, Artificial Sequence
  Peptide Marker for DYR1A_312_324
CQLGQRIYQYIQSG

SEQ ID NO: 18 - protein, 14 aa, Artificial Sequence
  Peptide Marker for EFS_246_258
GGTDEGIYDVPLLG

SEQ ID NO: 19 - protein, 13 aa, Artificial Sequence
  Peptide Marker for EGFR_1062_1074
EDSFLQRYSSDPT

SEQ ID NO: 20 - protein, 14 aa, Artificial Sequence
  Peptide Marker for EGFR_1103_1115
GSVQNPVYHNQPLG

SEQ ID NO: 21 - protein, 13 aa, Artificial Sequence
  Peptide Marker for EGFR_1118_1130
APSRDPHYQDPHS

SEQ ID NO: 22 - protein, 14 aa, Artificial Sequence
  Peptide Marker for EGFR_1165_1177
ISLDNPDYQQDFFG

SEQ ID NO: 23 - protein, 14 aa, Artificial Sequence
  Peptide Marker for EGFR_1190_1202
STAENAEYLRVAPG

SEQ ID NO: 24 - protein, 13 aa, Artificial Sequence
  Peptide Marker for EGFR_862_874
LGAEEKEYHAEGG

SEQ ID NO: 25 - protein, 13 aa, Artificial Sequence
  Peptide Marker for EGFR_908_920
MTFGSKPYDGIPA

SEQ ID NO: 26 - protein, 14 aa, Artificial Sequence
  Peptide Marker for ENOG_37_49
SGASTGIYEALELG

SEQ ID NO: 27 - protein, 14 aa, Artificial Sequence
  Peptide Marker for EPHA1_774_786
LDDFDGTYETQGGG

SEQ ID NO: 28 - protein, 14 aa, Artificial Sequence
  Peptide Marker for EPHA2_581_593
QLKPLKTYVDPHTG

SEQ ID NO: 29 - protein, 14 aa, Artificial Sequence
  Peptide Marker for EPHA2_765_777
EDDPEATYTTSGGG

SEQ ID NO: 30 - protein, 14 aa, Artificial Sequence
  Peptide Marker for EPHA4_589_601
LNQGVRTYVDPFTG

SEQ ID NO: 31 - protein, 14 aa, Artificial Sequence
  Peptide Marker for EPHA4_921_933
QAIKMDRYKDNFTG

SEQ ID NO: 32 - protein, 14 aa, Artificial Sequence
  Peptide Marker for EPHA7_607_619
TYIDPETYEDPNRG

SEQ ID NO: 33 - protein, 14 aa, Artificial Sequence
  Peptide Marker for EPHB1_771_783
DDTSDPTYTSSLGG

SEQ ID NO: 34 - protein, 14 aa, Artificial Sequence
  Peptide Marker for EPHB1_921_933
SAIKMVQYRDSFLG

SEQ ID NO: 35 - protein, 14 aa, Artificial Sequence
  Peptide Marker for EPHB4_583_595
IGHGTKVYIDPFTG

SEQ ID NO: 36 - protein, 14 aa, Artificial Sequence
  Peptide Marker for EPOR_361_373
SEHAQDTYLVLDKG

SEQ ID NO: 37 - protein, 14 aa, Artificial Sequence
  Peptide Marker for EPOR_419_431
ASAASFEYTILDPG

SEQ ID NO: 38 - protein, 14 aa, Artificial Sequence
  Peptide Marker for ERBB2_1241_1253
PTAENPEYLGLDVG

SEQ ID NO: 39 - protein, 13 aa, Artificial Sequence
  Peptide Marker for ERBB2_870_882
LDIDETEYHADGG

SEQ ID NO: 40 - protein, 13 aa, Artificial Sequence
  Peptide Marker for ERBB4_1181_1193
QALDNPEYHNASN

SEQ ID NO: 41 - protein, 14 aa, Artificial Sequence
  Peptide Marker for FABPH_13_25
DSKNFDDYMKSLGG

SEQ ID NO: 42 - protein, 14 aa, Artificial Sequence
  Peptide Marker for FAK1_569_581
RYMEDSTYYKASKG

SEQ ID NO: 43 - protein, 14 aa, Artificial Sequence
  Peptide Marker for FAK2_572_584
RYIEDEDYYKASVG

SEQ ID NO: 44 - protein, 14 aa, Artificial Sequence
  Peptide Marker for FER_707_719
RQEDGGVYSSSGLG

SEQ ID NO: 45 - protein, 14 aa, Artificial Sequence
  Peptide Marker for FES_706_718
REEADGVYAASGGG

SEQ ID NO: 46 - protein, 14 aa, Artificial Sequence
  Peptide Marker for FGFR2_762_774
TLTTNEEYLDLSQG

SEQ ID NO: 47 - protein, 14 aa, Artificial Sequence
  Peptide Marker for FGFR3_641_653
DVHNLDYYKKTTNG

SEQ ID NO: 48 - protein, 14 aa, Artificial Sequence
  Peptide Marker for FGFR3_753_765
TVTSTDEYLDLSAG

SEQ ID NO: 49 - protein, 14 aa, Artificial Sequence
  Peptide Marker for FRK_380_392
KVDNEDIYESRHEG

SEQ ID NO: 50 - protein, 14 aa, Artificial Sequence
  Peptide Marker for INSR_1348_1360
SLGFKRSYEEHIPG

SEQ ID NO: 51 - protein, 14 aa, Artificial Sequence
  Peptide Marker for INSR_992_1004
YASSNPEYLSASDG

SEQ ID NO: 52 - protein, 14 aa, Artificial Sequence
  Peptide Marker for JAK1_1015_1027
AIETDKEYYTVKDG

SEQ ID NO: 53 - protein, 14 aa, Artificial Sequence
  Peptide Marker for K2C6B_53_65
GAGFGSRSLYGLGG

SEQ ID NO: 54 - protein, 14 aa, Artificial Sequence
  Peptide Marker for KSYK_518_530
ALRADENYYKAQTG

SEQ ID NO: 55 - protein, 14 aa, Artificial Sequence
  Peptide Marker for LAT_194_206
MESIDDYVNVPESG

SEQ ID NO: 56 - protein, 14 aa, Artificial Sequence
  Peptide Marker for LAT_249_261
EEGAPDYENLQELG

SEQ ID NO: 57 - protein, 14 aa, Artificial Sequence
  Peptide Marker for LCK_387_399
RLIEDNEYTAREGG

SEQ ID NO: 58 - protein, 14 aa, Artificial Sequence
  Peptide Marker for MBP_198_210
ARTAHYGSLPQKSG

SEQ ID NO: 59 - protein, 14 aa, Artificial Sequence
  Peptide Marker for MBP_259_271
FGYGGRASDYKSAG

SEQ ID NO: 60 - protein, 14 aa, Artificial Sequence
  Peptide Marker for MBP_263_275
GRASDYKSAHKGFG

SEQ ID NO: 61 - protein, 14 aa, Artificial Sequence
  Peptide Marker for MET_1227_1239
RDMYDKEYYSVHNG

SEQ ID NO: 62 - protein, 13 aa, Artificial Sequence
  Peptide Marker for MK01_180_192
HTGFLTEYVATRW

SEQ ID NO: 63 - protein, 13 aa, Artificial Sequence
  Peptide Marker for MK01_198_210
IMLNSKGYTKSID

SEQ ID NO: 64 - protein, 14 aa, Artificial Sequence
  Peptide Marker for MK07_211_223
AEHQYFMTEYVATG

SEQ ID NO: 65 - protein, 14 aa, Artificial Sequence
  Peptide Marker for MK10_216_228
TSFMMTPYVVTRYG

SEQ ID NO: 66 - protein, 14 aa, Artificial Sequence
  Peptide Marker for MK12_178_190
ADSEMTGYVVTRWG

SEQ ID NO: 67 - protein, 14 aa, Artificial Sequence
  Peptide Marker for MK14_173_185
RHTDDEMTGYVATG

SEQ ID NO: 68 - protein, 14 aa, Artificial Sequence
  Peptide Marker for NTRK1_489_501
HIIENPQYFSDACG

SEQ ID NO: 69 - protein, 14 aa, Artificial Sequence
  Peptide Marker for NTRK2_509_521
PVIENPQYFGITNG

SEQ ID NO: 70 - protein, 14 aa, Artificial Sequence
  Peptide Marker for P85A_600_612
NENTEDQYSLVEDG

SEQ ID NO: 71 - protein, 14 aa, Artificial Sequence
  Peptide Marker for PAXI_111_123
VGEEEHVYSFPNKG

SEQ ID NO: 72 - protein, 14 aa, Artificial Sequence
  Peptide Marker for PAXI_24_36
FLSEETPYSYPTGG

SEQ ID NO: 73 - protein, 13 aa, Artificial Sequence
  Peptide Marker for PDPK1_2_14
ARTTSQLYDAVPI

SEQ ID NO: 74 - protein, 13 aa, Artificial Sequence
  Peptide Marker for PDPK1_369_381
DEDCYGNYDNLLS

SEQ ID NO: 75 - protein, 14 aa, Artificial Sequence
  Peptide Marker for PECA1_706_718
KKDTETVYSEVRKG

SEQ ID NO: 76 - protein, 14 aa, Artificial Sequence
  Peptide Marker for PERI_458_470
QRSELDKSSAHSYG

SEQ ID NO: 77 - protein, 14 aa, Artificial Sequence
  Peptide Marker for PGFRB_1002_1014
LDTSSVLYTAVQPG

SEQ ID NO: 78 - protein, 14 aa, Artificial Sequence
  Peptide Marker for PGFRB_572_584
VSSDGHEYIYVDPG

SEQ ID NO: 79 - protein, 14 aa, Artificial Sequence
  Peptide Marker for PGFRB_709_721
RPPSAELYSNALPG

SEQ ID NO: 80 - protein, 14 aa, Artificial Sequence
  Peptide Marker for PGFRB_768_780
SSNYMAPYDNYVPG

SEQ ID NO: 81 - protein, 14 aa, Artificial Sequence
  Peptide Marker for PGFRB_771_783
YMAPYDNYVPSAPG

SEQ ID NO: 82 - protein, 14 aa, Artificial Sequence
  Peptide Marker for PLCG1_1246_1258
EGSFESRYQQPFEG

SEQ ID NO: 83 - protein, 14 aa, Artificial Sequence
  Peptide Marker for PLCG1_764_776
IGTAEPDYGALYEG

SEQ ID NO: 84 - protein, 13 aa, Artificial Sequence
  Peptide Marker for PLCG1_776_788
EGRNPGFYVEANP

SEQ ID NO: 85 - protein, 14 aa, Artificial Sequence
  Peptide Marker for PP2AB_297_309
EPHVTRRTPDYFLG

SEQ ID NO: 86 - protein, 14 aa, Artificial Sequence
  Peptide Marker for PRRX2_202_214
WTASSPYSTVPPYG

SEQ ID NO: 87 - protein, 14 aa, Artificial Sequence
  Peptide Marker for RASA1_453_465
TVDGKEIYNTIRRG

SEQ ID NO: 88 - protein, 14 aa, Artificial Sequence
  Peptide Marker for RBL2_99_111
VPTVSKGTVEGNYG

SEQ ID NO: 89 - protein, 14 aa, Artificial Sequence
  Peptide Marker for RET_1022_1034
TPSDSLIYDDGLSG

SEQ ID NO: 90 - protein, 14 aa, Artificial Sequence
  Peptide Marker for RET_680_692
AQAFPVSYSSSGAG

SEQ ID NO: 91 - protein, 14 aa, Artificial Sequence
  Peptide Marker for RON_1346_1358
SALLGDHYVQLPAG

SEQ ID NO: 92 - protein, 14 aa, Artificial Sequence
  Peptide Marker for RON_1353_1365
YVQLPATYMNLGPG

SEQ ID NO: 93 - protein, 14 aa, Artificial Sequence
  Peptide Marker for SRC8_CHICK_470_482
VSQREAEYEPETVG

SEQ ID NO: 94 - protein, 14 aa, Artificial Sequence
  Peptide Marker for SRC8_CHICK_476_488
EYEPETVYEVAGAG

SEQ ID NO: 95 - protein, 14 aa, Artificial Sequence
  Peptide Marker for SRC8_CHICK_492_504
YQAEENTYDEYENG

SEQ ID NO: 96 - protein, 14 aa, Artificial Sequence
  Peptide Marker for STA5A_687_699
LAKAVDGYVKPQIG

SEQ ID NO: 97 - protein, 14 aa, Artificial Sequence
  Peptide Marker for STAT1_694_706
DGPKGTGYIKTELG

SEQ ID NO: 98 - protein, 14 aa, Artificial Sequence
  Peptide Marker for STAT3_698_710
DPGSAAPYLKTKFG

SEQ ID NO: 99 - protein, 14 aa, Artificial Sequence
  Peptide Marker for STAT4_686_698
TERGDKGYVPSVFG

SEQ ID NO: 100 - protein, 14 aa, Artificial Sequence
  Peptide Marker for STAT4_714_726
PSDLLPMSPSVYAG

SEQ ID NO: 101 - protein, 14 aa, Artificial Sequence
  Peptide Marker for TEC_512_524
RYFLDDQYTSSSGG

SEQ ID NO: 102 - protein, 14 aa, Artificial Sequence
  Peptide Marker for TYRO3_679_691
KIYSGDYYRQGCAG

SEQ ID NO: 103 - protein, 13 aa, Artificial Sequence
  Peptide Marker for VGFR1_1049_1061
KNPDYVRKGDTRL

SEQ ID NO: 104 - protein, 13 aa, Artificial Sequence
  Peptide Marker for VGFR2_1052_1064
DIYKDPDYVRKGD

SEQ ID NO: 105 - protein, 14 aa, Artificial Sequence
  Peptide Marker for VGFR3_1061_1073
DIYKDPDYVRKGSG

SEQ ID NO: 106 - protein, 14 aa, Artificial Sequence
  Peptide Marker for VINC_815_827
KSFLDSGYRILGAG

SEQ ID NO: 107 - protein, 14 aa, Artificial Sequence
  Peptide Marker for ZAP70_485_497
ALGADDSYYTARSG

SEQ ID NO: 108 - protein, 14 aa, Artificial Sequence
  Peptide Marker for ZBT16_621_633
LRTHNGASPYQCTG

(19) United States
(12) Patent Application Publication
(10) Pub. No.: US 2013/0217055 A1
(43) Pub. Date: Aug. 22, 2013

(54) METHOD FOR PREDICTING TYROSINE KINASE INHIBITOR (TKI) RESISTANCE IN PATIENTS SUFFERING FROM CHRONIC MYELOGENOUS LEUKEMIA (CML)

(71) Applicants: Pieter Jacob Boender, Nijmegen (NL); Adriana Van Den Berg, Geldrop (NL); Robby Ruijtenbeek, Utrecht (NL); Jeroen Johannes Wilhelmus Maria Janssen, Hilversum (NL); Gerrit Johan Ossenkoppele, Uithoorn (NL)

(72) Inventors: Pieter Jacob Boender, Nijmegen (NL); Adriana Van Den Berg, Geldrop (NL); Robby Ruijtenbeek, Utrecht (NL); Jeroen Johannes Wilhelmus Maria Janssen, Hilversum (NL); Gerrit Johan Ossenkoppele, Uithoorn (NL)

(73) Assignee: PamGene B.V., 's-Hertogenbosch (NL)

(21) Appl. No.: 13/879,046

(22) PCT Filed: Oct. 17, 2011
(86) PCT No.: PCT/EP2011/068128

§ 371 Date: Apr. 12, 2013

(30) Foreign Application Priority Data
Oct. 15, 2010 (EP) 10187760.3

(51) Int. Cl. C12Q 1/48

(52) U.S. Cl. CPC C12Q 1/485
USPC 435/15

(57) ABSTRACT

The present invention relates to a method for determining or predicting the response of a patient diagnosed with chronic myelogenous leukaemia (CML) to treatment with a tyrosine kinase inhibitor. More specifically, the present invention provides methods which measure kinase activity by studying phosphorylation levels and profiles and inhibitions thereof thereby diagnosing CML patients resistant to treatment with Imatinib.

### FIELD OF THE INVENTION

The present invention relates to a method for diagnosing a patient with chronic myelogeneous leukaemia (CML) and the present invention relates to a method for determining or predicting the response prior to treatment of a patient diagnosed with chronic myelogenous leukaemia (CML) to treatment with a tyrosine kinase inhibitor. More specifically, the present invention provides methods which measure kinase activity by studying phosphorylation levels and profiles and inhibitions thereof thereby predicting existing and future resistance of CML patients to treatment with Imatinib.

### BACKGROUND OF THE INVENTION

Chronic myelogenous (or myeloid) leukemia (CML), also known as chronic granulocytic leukemia (CGL), is a cancer of the white blood cells. It is a form of leukemia characterized by the increased and unregulated growth of predominantly myeloid cells in the bone marrow and the accumulation of these cells in the blood. CML is a clonal bone marrow stem cell disorder in which proliferation of mature granulocytes (neutrophils, eosinophils, and basophils) and their precursors is the main finding. It is a type of myeloproliferative disease associated with a characteristic chromosomal translocation called the Philadelphia chromosome. At present CML is treated with Imatinib, which have dramatically improved the survival. The success of treatment with Imatinib in the majority of chronic phase CML patients is well documented. Imatinib treatment can induce a complete or nearly complete cytogenetic remission in up to 80 percent of patients when they are treated in chronic phase.

However, in 20 percent of CML patients, this treatment is inefficient. Indeed, about 20% of CML patients treated with Imatinib are or become resistant to the treatment. Two kinds of resistances can be distinguished: in primary resistance, patients never respond to treatment; in secondary resistance, patients first respond to treatment, but after a few months or years, they relapse. In some cases, mutation in the BCR-ABL tyrosine kinase domain can explain a newly occurring resistance. However, in other cases, no mutation allows to explain the resistance phenomenon.

At present there are however no clinical or analytical tools are available to make the distinction between responders and non-responders prior to deciding which treatment to administer. A few attempts of predictive biomarkers have been described in the literature. These methods provide a considerable variability, and may not always be considered as being very robust. Furthermore, some of these methods only provide a poor predictivity. This is especially due to the fact that these screenings are based on the detection of a limited number of proteins or genes. A small variation in the gene or protein expression may have profound effects on the screening method. In this context, there is a strong need for a predictive tool which could help physicians to predict the response of a given CML patient to treatment with Imatinib, and/or to detect a secondary resistance at a very early stage, so that the treatment of said patient can be adapted.

In view of the above, there remains a pressing need for methods that provide a fast and accurate prediction of the response of a patient diagnosed with CML to treatment with a TKI in general, and Imatinib treatment specifically.

The present invention aims at providing methods and devices for predicting the response of a patient diagnosed with CML to treatment with a TKI in general, and Imatinib treatment specifically. The present invention also aims to provide methods and devices for predicting the resistance to TKI treatment of patients diagnosed with CML. The method of the present invention therefore adds to the existing assays currently used to select therapies in CML patients.

### SUMMARY OF THE INVENTION

The present invention provides methods and devices that enable the determination of the response of a patient diagnosed with chronic myelogenous leukaemia (CML), to a tyrosine kinase inhibitor (TKI) by measuring kinase activity of a sample, obtained from said patient diagnosed with CML. The present invention further shows how the method and devices can be used to predict the resistance of patients diagnosed with CML to treatment with TKI and preferably treatment with Imatinib. The method of the present invention therefore adds to the existing assays currently used to select therapies in CML patients. Preferably, in one embodiment of the present invention, methods are provided wherein the kinase activity is protein kinase activity. For purposes of the present invention, and as used herein the term “enzyme activity”, “kinase activity” or “protein kinase activity” refer to the formation of reaction product(s) by a certain amount of enzyme, kinase or protein kinase acting on a substrate during the course of the assay.

The present invention therefore provides a method for predicting the response of a patient diagnosed with chronic myelogenous leukaemia (CML), to a tyrosine kinase inhibitor (TKI), comprising the steps of:

(a) measuring the kinase activity of a sample, obtained from said patient diagnosed with CML, by contacting said sample with at least one protein kinase substrate, thereby providing a phosphorylation profile of said sample, said phosphorylation profile comprising the phosphorylation levels of phosphorylation sites present in at least 10 peptide markers as listed in Table 1; and,
(b) determining from said phosphorylation profile the response of said patient to said TKI.

Preferably the present invention provides a method for predicting the response of a patient diagnosed with chronic myelogenous leukaemia (CML), to a tyrosine kinase inhibitor (TKI), comprising the steps of:

(a) measuring the kinase activity of a sample, obtained from said patient diagnosed with CML, by contacting said sample with immobilized protein kinase substrates, thereby providing a phosphorylation profile of said sample, said phosphorylation profile comprising the phosphorylation levels of phosphorylation sites present in at least 10 peptide markers as listed in Table 1; and,
(b) determining from said phosphorylation profile the response of said patient to said TKI.

In a particular embodiment, the present invention relates to a method according to the present invention wherein said TKI is Imatinib.

Another embodiment of the present invention regards a method for diagnosing CML for patients, wherein the kinase activity of a sample, obtained from said patient, is measured, wherein said kinase activity measurement provides phosphorylation profiles of said sample thereby diagnosing CML. In a particular embodiment said patients are expected to have CML.

In a further embodiment, the present invention relates to a method for diagnosing CML for patients according to the present invention wherein said phosphorylation profiles comprise the phosphorylation levels of phosphorylation sites present in at least 10 peptide markers as listed in Table 1. In a particular embodiment said patients are expected to have CML.

Another embodiment of the present invention regards a computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method according to the present invention.

The present invention also relates in another embodiment to a kit for predicting the response of a patient diagnosed with chronic myelogenous leukaemia (CML), to a tyrosine kinase inhibitor (TKI), comprising at least one array comprising immobilized proteins, peptides or peptide mimetics comprising phosphorylation sites present in at least 10 peptide markers as listed in Table 1, and optionally a computer readable storage medium having recorded thereon one or more programs for carrying out the method according to the present invention.

These and further aspects and embodiments are described in the following sections and in the claims.

### DETAILED DESCRIPTION OF THE INVENTION

Before the present method and devices used in the invention are described, it is to be understood that this invention is not limited to particular methods, components, or devices described, as such methods, components, and devices may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The terms “comprising”, “comprises” and “comprised of also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

The present invention provides methods and devices that enable the prediction or determination of the response of a patient diagnosed with chronic myelogenous leukaemia (CML), to a tyrosine kinase inhibitor (TKI) by measuring kinase activity of a sample, obtained from said patient diagnosed with CML. The present invention further shows how the method and devices can be used to predict the resistance of patients diagnosed with CML to treatment or therapy with TKI and preferably treatment with Imatinib. The method of the present invention therefore adds to the existing assays currently used to select therapies in CML patients. Preferably, in one embodiment of the present invention, methods are provided wherein the kinase activity is protein kinase activity. For purposes of the present invention, and as used herein the term “enzyme activity”, “kinase activity” or “protein kinase activity” refer to the formation of reaction product(s) by a certain amount of enzyme, kinase or protein kinase acting on a substrate during the course of the assay.

The method according to the present invention can be used to predict or assess both primary and secondary resistance to TKI treatment. With primary resistance is meant resistance in patients that never respond to TKI treatment. In secondary resistance is meant resistance in patients which first respond to TKI treatment, but after a few months or years, resistance occurs.

Protein kinase activity is referred to as the activity of protein kinases. A protein kinase is a generic name for all enzymes that transfer a phosphate to a protein. About three to four percent of the human genome contains transcription information for the formation of protein kinases. Currently, there are about 518 known different protein kinases. However, because three to four percent of the human genome is a code for the formation of protein kinases, there may be many more separate kinases in the human body.

A protein kinase is a kinase enzyme that modifies other proteins by covalently coupling phosphate groups to them. This process or activity is also referred to as phosphorylation. Phosphorylation can therefore be regarded as the process of the addition of a phosphate group to a substrate. Phosphorylation usually results in a functional change of the substrate by changing enzyme activity, cellular location, or association with other proteins. Up to 30% of all proteins may be modified by kinase activity, and kinases are known to regulate the majority of cellular pathways, especially those involved in signal transduction, the transmission of signals within the cell. The chemical activity of a kinase involves removing a phosphate group from ATP or GTP and covalently attaching it to amino acids such as serine, threonine, tyrosine, histidine, aspartic acid and/or glutamic acid that have a free hydroxyl group. Most known kinases act on both serine and threonine, others act on tyrosine, and a number act on all serine, threonine and tyrosine. The protein kinase activity monitored with the method of the present invention is preferably directed to protein kinases acting towards serine, threonine and/or tyrosine, preferably acting on both serine and threonine, on tyrosine or on serine, threonine and tyrosine and more preferably the method of the present invention if preferably directed to protein kinases acting towards tyrosines.

Protein kinases are distinguished by their ability to phosphorylate substrates on discrete sequences. These sequences have been determined by sequencing the amino acids around the phosphorylation sites and are usually distinct for each protein kinase. The recognition sequence on each substrate is specific for each kinase catalyst.

Because protein kinases have profound effects on a cell, their activity is highly regulated. Kinases are turned on or off by for instance phosphorylation, by binding of activator proteins or inhibitor proteins, or small molecules, or by controlling their location in the cell relative to their substrates. Deregulated kinase activity is a frequent cause of disease, particularly cancer, where kinases regulate many aspects that control cell growth, movement and death. Therefore monitoring the protein kinase activity in tissues can be of great importance and a large amount of information can be obtained when comparing the kinase activity of different tissue samples.

As described in the present invention, the inventors have surprisingly found that the response of a patient diagnosed with CML to targeted treatment with a TKI, preferably Imatinib, can be predicted and/or determined on the basis of the measurement of the kinase activity of a sample obtained from said patient diagnosed with CML.

The measurement of the kinase activity is performed by contacting a sample, obtained from said patient diagnosed with CML, with one or more substrates, preferably protein kinase substrates, thereby generating a phosphorylation profile.

Said protein kinase substrates as used herein, are preferably peptides, proteins or peptide mimetics. The protein kinase substrates each comprise, preferably one or more, phosphorylation sites that can be phosphorylated by the protein kinases present in the sample. Therefore, exposure of a protein kinase substrate to a sample comprising a protein kinase results in the phosphorylation of one or more of the phosphorylation sites of the protein kinase substrate. This phosphorylation activity can be measured using techniques known in the art. Therefore, during the measurement method the kinase enzymes present in the sample will phosphorylate, preferably one or more, of the phosphorylation sites on one or more protein kinase substrates. The inventors have observed essential differences between kinase activity of samples obtained from a TKI resistant and a non-TKI-resistant CML patient. Consequently, the inventors have observed that the kinases present in a sample from a CML patient will phosphorylate protein kinase substrates differently depending on the response to treatment with a TKI, and preferably treatment with Imatinib. Phosphorylation signals differ between the samples, resulting in phosphorylation patterns that differ depending on response to treatment with a TKI, and preferably treatment with Imatinib.

For purposes of the present invention, and as used herein the term “treatment” refers to the use of a pharmaceutical drug, also referred to as medicine or medicament, preferably a TKI and more preferably Imatinib, wherein said treatment is intended for use in the diagnosis, cure, mitigation, treatment, or prevention of CML.

As used herein “tyrosine kinase inhibitor” or “TKI” refers to tyrosine kinase inhibitor molecules that in most cases compete for binding with ATP, the natural substrate of kinases. TKI thus bind to tyrosine kinases and reduce or inhibit the kinase activity. More particularly TKI refers to a type of enzyme inhibitor which blocks the action of one or more tyrosine protein kinases. In a particular embodiment according to the present invention said TKI is chosen from Imatinib, nilotinib, dasatinib or bosutinib and preferably said TKI is Imatinib.

The present invention therefore provides a method for predicting the response of a patient diagnosed with chronic myelogenous leukaemia (CML), to a tyrosine kinase inhibitor (TKI), comprising the steps of:

(a) measuring the kinase activity of a sample, obtained from said patient diagnosed with CML, by contacting said sample with at least one protein kinase substrate, thereby providing a phosphorylation profile of said sample, said phosphorylation profile comprising the phosphorylation levels of phosphorylation sites present in at least 10 peptide markers as listed in Table 1; and,
(b) determining from said phosphorylation profile the response of said patient to said TKI.

Preferably the present invention provides a method for predicting the response of a patient diagnosed with chronic myelogenous leukaemia (CML), to a tyrosine kinase inhibitor (TKI), comprising the steps of:

(a) measuring the kinase activity of a sample, obtained from said patient diagnosed with CML, by contacting said sample with immobilized protein kinase substrates, thereby providing a phosphorylation profile of said sample, said phosphorylation profile comprising the phosphorylation levels of phosphorylation sites present in at least 10 peptide markers as listed in Table 1; and,
(b) determining from said phosphorylation profile the response of said patient to said TKI.

As referred to in the present application chronic myelogenous or myeloid leukemia (CML), also known as chronic granulocytic leukemia (CGL), regards a cancer of the white blood cells. It is a form of leukemia characterized by the increased and unregulated growth of predominantly myeloid cells in the bone marrow and the accumulation of these cells in the blood. CML is a clonal bone marrow stem cell disorder in which proliferation of mature granulocytes (neutrophils, eosinophils, and basophils) and their precursors is the main finding.

As used in the present invention, the term “sample” refers to a sample obtained from an organism (patient) such as human or from components (e.g. tissue or cells) of such an organism. Said sample is preferably obtained from a patient diagnosed with CML and are preferably derived from the blood or bone marrow of said patient. Said sample is thereby referred to as a ‘clinical sample’ which is a sample derived from a CML patient.

In a preferred embodiment of the present invention said sample is a sample that has undergone a preparation step prior to the steps according to the method of the present invention. Preferably said preparation step is a step where the protein kinases present in said sample are released by lysis. Additionally the kinases in the sample may be stabilized, maintained, enriched or isolated, and the measurement of the kinase activity as performed in step (a) occurs on the enriched or isolated protein kinase sample. By first enriching protein kinases in the sample or isolating protein kinases from the sample the subsequent measurement of the kinase activity will occur in a more efficient and reliable manner. Also the clarity and intensity of the obtained phosphorylation signal will be increased as certain contaminants are being removed during the enriching or isolating step.

As used in the present invention, the term “phosphorylation profile” refers to a data set representative for the phosphorylation levels of, preferably one or more, phosphorylation sites present on the protein kinase substrates. When measuring the kinase activity of a sample by contacting said sample with protein kinase substrates a specific phosphorylation profile is obtained. The phosphorylation profile is generated by the phosphorylation of the protein kinase substrates with the protein kinases present in the sample and it comprises the level of phosphorylation of the phosphorylation sites present on the protein kinase substrates used. A phosphorylation profile can thus be generated when using at least one protein kinase substrate in different test conditions such as for example by comparing the phosphorylation of a sample on one peptide or protein (protein kinase substrate) in the presence and absence of a protein kinase inhibitor. More frequently phosphorylation profiles of a sample will be measured using several protein kinase substrates in the same or sequentially carried out experiments. Preferably, the present invention determines tyrosine kinase activity levels or profiles.

It should be noted that a person skilled in the art will appreciate that the methods of the present invention can use phosphorylation profiles as a basis for determining and predicting the response to TKI, an preferably Imatinib, of a patient suffering from CML. However, the phosphorylation levels of individual protein kinase substrates can also be used as a basis for determining or predicting the resistance of said CML patient to TKI and preferably Imatinib.

It should be noted that for the measurement of the protein kinase activity, ATP or any other phosphate source needs to be added to the sample when it is contacted with the protein kinase substrates. The presence of ATP will lead to a phosphorylation of the protein kinase substrates. Alternatively, the phosphorylation of the protein kinase substrates can be performed in the absence of exogenous ATP. When no ATP is added during the incubation of the sample with the protein kinase substrates, the endogenous ATP, the ATP naturally present in the sample, will act as the primary source of ATP.

The phosphorylation level of each of the protein kinase substrates can be monitored using any method known in the art. The response of the protein kinase substrates is determined using a detectable signal, said signal resulting from the interaction of the sample with the protein kinase substrates or by for instance measuring mass differences using mass spectrometry. In determining the interaction of the sample with the protein kinase substrates the signal is the result of the interaction of the phosphorylated substrates with a molecule capable of binding to the phosphorylated substrates. This binding can be detected by e.g. surface plasmon resonance or by the molecule being detectably labelled. For the latter, the molecule that specifically binds to the substrates of interest (e.g. antibody or polynucleotide probe) can be detectably labelled by virtue of containing an atom (e.g. radionuclide), molecule (e.g. fluorescein), or enzyme or particle or complex that, due to a physical or chemical property, indicates the presence of the molecule. A molecule may also be detectably labelled when it is covalently bound to or otherwise associated with a “reporter” molecule (e.g. a biomolecule such as an enzyme) that acts on a substrate to produce a detectable atom, molecule or other complex.

Detectable labels suitable for use in the present invention include any composition detectable by spectroscopic, photochemical, biochemical, immunochemical, electrical, optical or chemical means. Labels useful in the present invention include biotin for staining with labelled avidin or streptavidin conjugate, magnetic beads (e.g. Dynabeads'), fluorescent dyes (e.g. fluorescein, fluorescein-isothiocyanate (FITC), Texas red, rhodamine, green fluorescent protein, enhanced green fluorescent protein and related proteins with other fluorescence emission wavelengths, lissamine, phycoerythrin, Cy2, Cy3, Cy3.5, Cy5, Cy5.5, Cy7, FluorX [Amersham], SYBR Green I & II [Molecular Probes], and the like), radiolabels (e.g. 3H, 125I, 35S, 14C, or 32P), enzymes (e.g. hydrolases, particularly phosphatases such as alkaline phosphatase, esterases and glycosidases, or oxidoreductases, particularly peroxidases such as horse radish peroxidase, and the like), substrates, cofactors, chemilluminescent groups, chromogenic agents, and colorimetric labels such as colloidal gold or coloured glass or plastic (e.g. polystyrene, polypropylene, latex, etc.), protein particles or beads.

Means of detecting such labels are well known to those of skill in the art. Thus, for example, chemiluminescent and radioactive labels may be detected using photographic film or scintillation counters, and fluorescent markers may be detected using a photodetector to detect emitted light (e.g. as in fluorescence-activated cell sorting). Enzymatic labels are typically detected by providing the enzyme with a substrate and detecting a coloured reaction product produced by the action of the enzyme on the substrate. Colorimetric labels are detected by simply visualizing the coloured label. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple colorimetric labels may be detected by observing the colour associated with the label. Fluorescence resonance energy transfer has been adapted to detect binding of unlabeled ligands, which may be useful on arrays.

In a particular embodiment of the present invention the response of the protein kinase substrates to the sample is determined using detectably labelled antibodies; more in particular fluorescently labelled antibodies. In those embodiments of the invention where the substrates consist of protein kinase substrates, the response of the protein kinase substrates is determined using fluorescently labelled anti-phosphotyrosine antibodies, fluorescently labelled anti-phosphoserine or fluorescently labelled anti-phosphothreonine antibodies. The use of fluorescently labelled anti-phosphotyrosine antibodies or fluorescently labelled anti-phosphoserine or fluorescently labelled anti-phosphothreonine antibodies in the method of the present invention, allows real-time or semi real-time determination of the protein kinase activity and accordingly provides the possibility to express the protein kinase activity as the initial velocity of protein kinase derived from the activity over a certain period of incubation of the sample on the protein kinase substrates.

In addition, because the phosphorylation profile is generated by generating and comparing the phosphorylation levels of a number of protein kinase substrates, the phosphorylation profile is surprisingly found to be less affected by variation, for example biological variation, experimental variation, compared to other types of profiles. This provides a more robust, more sensitive, more reproducible and more reliable method for determining the expected response to TKI treatment.

The inventors have surprisingly found that kinase activity measurements enable the early prediction of the response of CML patients to TKI treatment. This prediction provides information on the efficacy of the TKI treatment.

According to the present invention, the phosphorylation profiles comprise the phosphorylation levels of, preferably 10 or more, phosphorylation sites present in at least any of the peptide markers as listed in table 1. Preferably phosphorylation levels will be studied of phosphorylation sites present in at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95, 100, 105, or 108 of the peptide markers listed in Table 1.

The term “peptide markers” in the context of the present invention refers to the fact that the peptides as listed in Table 1 can be preferably used according to the methods of the present invention to measure the phosphorylation levels of phosphorylation sites of said markers in the presence of protein kinase present in samples. The phosphorylation levels of the individual phosphorylation sites present in said markers may be measured and compared in different ways. Therefore the present invention is not limited to the use of peptides identical to any of these peptide markers as listed in Table 1 as such. The skilled person may easily on the basis of the peptide markers listed in Table 1 design variant peptides compared to the specific peptides in said Table and use such variant peptides in a method for measuring phosphorylation levels of phosphorylation sites common to said peptide markers as listed in Table 1. These variant peptides may have one or more (2, 3, 4, 5, 6, 7, etc.) amino acids more or less than the given peptides and may also have amino acid substitutions (preferably conservative amino acid substitutions) as long as these variant peptides retain at least one or more of the phosphorylation sites of said original peptides as listed in said table. Further the skilled person may also easily carry out the methods according to the present invention by using proteins (full length or N- or C-terminally truncated) comprising the amino acid regions of the “peptide markers” listed in Table 1 as sources for studying the phosphorylation of sites present in the amino acid regions of the peptides listed in Table 1. Also the skilled person may use peptide mimetics.

The protein kinase substrates as used in the methods described herein, are meant to include peptides, proteins or peptide mimetics comprising, preferably one or more, of the phosphorylation sites of the peptide markers of Table 1. Said one or more phosphorylation sites are specifically phosphorylated by the protein kinases present in the sample thereby providing a phosphorylation profile. More preferably the protein kinase substrates (peptides, proteins or peptide mimetics) as used in the method of the present invention comprise, preferably one or more, of the phosphorylation sites present in at least two peptide markers as listed in Table 1. More particularly said protein kinase substrates represent the one or more phosphorylation sites present in at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95, 100, 105 or 108 peptide markers as listed in Table 1. In a more preferred embodiment the protein kinase substrates comprise or consist of, preferably one or more, phosphorylation sites present in all of the peptide markers listed in Table 1.

A person skilled in the art will appreciate that the phosphorylation sites present in a single peptide marker as listed in Table 1 enable prediction of the resistance of a CML patient to TKI treatment, and more preferably to treatment with Imatinib. However, when the number of peptide markers as listed in Table 1 increases, so will increase the specificity, accuracy and sensitivity of the method according to the present invention. When for example only one protein kinase substrate comprising the phosphorylation sites of a single peptide marker as listed in table 1 is used for prediction the response of a CML patient to TKI treatment, the accuracy of the method will be lower, compared to a method where the prediction the response of a CML patient to TKI treatment uses multiple protein kinase substrates comprising the phosphorylation sites of multiple peptide markers as listed in table 1. The highest method accuracy will be obtained when all protein kinase substrates comprising the phosphorylation sites of all peptide markers as listed in table 1 are used.

TABLE 1
list of 108 peptide markers comprising phosphorylation sites used
for determining the kinase activity, their sequence and SEQ ID NO The
name of the peptide markers refers to the associated proteins and also
refers to the start and the end position of the amino acid sequence.

| SEQ ID NO | Name | Sequence (N terminus to C terminus) |
|---|---|---|
| 1 | ACHD_383_395 | YISKAEEYFLLKSG |
| 2 | AMPE_5_17 | EREGSKRYCIQTKG |
| 3 | ANXA1_14_26 | IENEEQEYVQTVKG |
| 4 | ANXA2_17_29 | HSTPPSAYGSVKAG |
| 5 | B3AT_39_51 | TEATATDYHTTSHG |
| 6 | CALM_93_105 | FDKDGNGYISAAEG |
| 7 | CALM_95_107 | KDGNGYISAAELRG |
| 8 | CBL_693_705 | EGEEDTEYMTPSSG |
| 9 | CD3Z_116_128 | KDKMAEAYSEIGMG |
| 10 | CD79A_181_193 | EYEDENLYEGLNLG |
| 11 | CDK2_8_20 | EKIGEGTYGWYKG |
| 12 | CDK3_12_26 | EGTYGVVYKAKNRETG |
| 13 | CTNB1_79_91 | VADIDGQYAMTRA |
| 14 | DCX_109_121 | GIVYAVSSDRFRSG |
| 15 | DDR1_506_518 | LLLSNPAYRLLLAG |
| 16 | DYR1A_212_224 | KHDTEMKYYIVHLG |
| 17 | DYR1A_312_324 | CQLGQRIYQYIQSG |
| 18 | EFS_246_258 | GGTDEGIYDVPLLG |
| 19 | EGFR_1062_1074 | EDSFLQRYSSDPT |
| 20 | EGFR_1103_1115 | GSVQNPVYHNQPLG |
| 21 | EGFR_1118_1130 | APSRDPHYQDPHS |
| 22 | EGFR_1165_1177 | ISLDNPDYQQDFFG |
| 23 | EGFR_1190_1202 | STAENAEYLRVAPG |
| 24 | EGFR_862_874 | LGAEEKEYHAEGG |
| 25 | EGFR_908_920 | MTFGSKPYDGIPA |
| 26 | ENOG_37_49 | SGASTGIYEALELG |
| 27 | EPHA1_774_786 | LDDFDGTYETQGGG |
| 28 | EPHA2_581_593 | QLKPLKTYVDPHTG |
| 29 | EPHA2_765_777 | EDDPEATYTTSGGG |
| 30 | EPHA4_589_601 | LNQGVRTYVDPFTG |
| 31 | EPHA4_921_933 | QAIKMDRYKDNFTG |
| 32 | EPHA7_607_619 | TYIDPETYEDPNRG |
| 33 | EPHB1_771_783 | DDTSDPTYTSSLGG |
| 34 | EPHB1_921_933 | SAIKMVQYRDSFLG |
| 35 | EPHB4_583_595 | IGHGTKVYIDPFTG |
| 36 | EPOR_361_373 | SEHAQDTYLVLDKG |
| 37 | EPOR_419_431 | ASAASFEYTILDPG |
| 38 | ERBB2_1241_1253 | PTAENPEYLGLDVG |
| 39 | ERBB2_870_882 | LDIDETEYHADGG |
| 40 | ERBB4_1181_1193 | QALDNPEYHNASN |
| 41 | FABPH_13_25 | DSKNFDDYMKSLGG |
| 42 | FAK1_569_581 | RYMEDSTYYKASKG |
| 43 | FAK2_572_584 | RYIEDEDYYKASVG |
| 44 | FER_707_719 | RQEDGGVYSSSGLG |
| 45 | FES_706_718 | REEADGVYAASGGG |
| 46 | FGFR2_762_774 | TLTTNEEYLDLSQG |
| 47 | FGFR3_641_653 | DVHNLDYYKKTTNG |
| 48 | FGFR3_753_765 | TVTSTDEYLDLSAG |
| 49 | FRK_380_392 | KVDNEDIYESRHEG |
| 50 | INSR_1348_1360 | SLGFKRSYEEHIPG |
| 51 | INSR_992_1004 | YASSNPEYLSASDG |
| 52 | JAK1_1015_1027 | AIETDKEYYTVKDG |
| 53 | K2C6B_53_65 | GAGFGSRSLYGLGG |
| 54 | KSYK_518_530 | ALRADENYYKAQTG |
| 55 | LAT_194_206 | MESIDDYVNVPESG |
| 56 | LAT_249_261 | EEGAPDYENLQELG |
| 57 | LCK_387_399 | RLIEDNEYTAREGG |
| 58 | MBP_198_210 | ARTAHYGSLPQKSG |
| 59 | MBP_259_271 | FGYGGRASDYKSAG |
| 60 | MBP_263_275 | GRASDYKSAHKGFG |
| 61 | MET_1227_1239 | RDMYDKEYYSVHNG |
| 62 | MK01_180_192 | HTGFLTEYVATRW |
| 63 | MK01_198_210 | IMLNSKGYTKSID |
| 64 | MK07_211_223 | AEHQYFMTEYVATG |
| 65 | MK10_216_228 | TSFMMTPYVVTRYG |
| 66 | MK12_178_190 | ADSEMTGYVVTRWG |
| 67 | MK14_173_185 | RHTDDEMTGYVATG |
| 68 | NTRK1_489_501 | HIIENPQYFSDACG |
| 69 | NTRK2_509_521 | PVIENPQYFGITNG |
| 70 | P85A_600_612 | NENTEDQYSLVEDG |
| 71 | PAXI_111_123 | VGEEEHVYSFPNKG |
| 72 | PAXI_24_36 | FLSEETPYSYPTGG |
| 73 | PDPK1_2_14 | ARTTSQLYDAVPI |
| 74 | PDPK1_369_381 | DEDCYGNYDNLLS |
| 75 | PECA1_706_718 | KKDTETVYSEVRKG |
| 76 | PERI_458_470 | QRSELDKSSAHSYG |
| 77 | PGFRB_1002_1014 | LDTSSVLYTAVQPG |
| 78 | PGFRB_572_584 | VSSDGHEYIYVDPG |
| 79 | PGFRB_709_721 | RPPSAELYSNALPG |
| 80 | PGFRB_768_780 | SSNYMAPYDNYVPG |
| 81 | PGFRB_771_783 | YMAPYDNYVPSAPG |
| 82 | PLCG1_1246_1258 | EGSFESRYQQPFEG |
| 83 | PLCG1_764_776 | IGTAEPDYGALYEG |
| 84 | PLCG1_776_788 | EGRNPGFYVEANP |
| 85 | PP2AB_297_309 | EPHVTRRTPDYFLG |
| 86 | PRRX2_202_214 | WTASSPYSTVPPYG |
| 87 | RASA1_453_465 | TVDGKEIYNTIRRG |
| 88 | RBL2_99_111 | VPTVSKGTVEGNYG |
| 89 | RET_1022_1034 | TPSDSLIYDDGLSG |
| 90 | RET_680_692 | AQAFPVSYSSSGAG |
| 91 | RON_1346_1358 | SALLGDHYVQLPAG |
| 92 | RON_1353_1365 | YVQLPATYMNLGPG |
| 93 | SRC8_CHICK_470_482 | VSQREAEYEPETVG |
| 94 | SRC8_CHICK_476_488 | EYEPETVYEVAGAG |
| 95 | SRC8_CHICK_492_504 | YQAEENTYDEYENG |
| 96 | STA5A_687_699 | LAKAVDGYVKPQIG |
| 97 | STAT1_694_706 | DGPKGTGYIKTELG |
| 98 | STAT3_698_710 | DPGSAAPYLKTKFG |
| 99 | STAT4_686_698 | TERGDKGYVPSVFG |
| 100 | STAT4_714_726 | PSDLLPMSPSVYAG |
| 101 | TEC_512_524 | RYFLDDQYTSSSGG |
| 102 | TYRO3_679_691 | KIYSGDYYRQGCAG |
| 103 | VGFR1_1049_1061 | KNPDYVRKGDTRL |
| 104 | VGFR2_1052_1064 | DIYKDPDYVRKGD |
| 105 | VGFR3_1061_1073 | DIYKDPDYVRKGSG |
| 106 | VINC_815_827 | KSFLDSGYRILGAG |
| 107 | ZAP70_485_497 | ALGADDSYYTARSG |
| 108 | ZBT16_621_633 | LRTHNGASPYQCTG |

It should further be noted that according to a preferred embodiment of the present invention the peptide markers as listed in Table 1 can be used as such for carrying out the methods according to the present invention. The present invention however also includes the use of analogs and combinations of these peptide markers for use in the method according to the present invention. The peptide marker analogs include peptide markers which show a sequence identity of more than 70%, preferably more than 80% and more preferably more than 90%.

In a particular embodiment, the present invention relates to a method according to the present invention wherein said TKI is Imatinib.

In yet another embodiment, the present invention relates to a method according to the present invention wherein step (b) is replaced by steps (c) and (d) as provided below. The method according to the present invention may therefore comprise the steps of:

(a) measuring the kinase activity of a sample, obtained from said patient diagnosed with CML, by contacting said sample with at least one protein kinase substrate, thereby providing a phosphorylation profile of said sample, said phosphorylation profile comprising the phosphorylation levels of phosphorylation sites present in at least 10 peptide markers as listed in Table 1;
(c) calculating a classifier parameter from said phosphorylation profile; and;
(d) predicting or determining the expected response or resistance of said patient to treatment with said TKI on the basis of said classifier parameter.

By establishing a classifier parameter for determining the prediction of the response of the CML patient to TKI treatment, the method of the present invention provides a criterion for analysing the results obtained from the method of the present invention. This criterion enables a person to provide a prediction of therapy efficacy, diagnosis or prognosis on the basis of a single or limited number of data. The person providing the prediction of therapy, diagnosis or prognosis does not have to interpret an entire set of data, but rather bases his conclusion on the basis of a single or limited number of criteria.

The term “classifier parameter” as used herein represents a discriminating value which has been determined by establishing the phosphorylation profile for from TKI resistant and non-TKI-resistant CML patient. Said discriminating value identifies the prediction of the expected response of the TKI treatment of CML patients. The classifier parameter includes information regarding the phosphorylation level of several protein kinase substrates. Classification is a procedure in which individual items are placed into groups based on quantitative information on one or more characteristics inherent in the items (e.g. phosphorylation levels or profiles of a sample) and based on a training set of previously labelled items. The classifier parameter is calculated by applying a “classifier” to the measured phosphorylation levels of a sample. Based on the classifying parameter a sample is assigned to (or predicted to belong to) a class (predicting response of said patient to the treatment). The classifier has been previously determined by comparing samples which are known to belong to the respective relevant classes. For instance the classifier may be a mathematical function that uses information regarding the phosphorylation level of several protein kinase substrates which individual protein kinase substrates can be statistically weighted based on the measured phosphorylation level of a number of protein kinase substrates (or values derived from that). Several methods are known in the art for developing a classifier including the neural network (Multi-layer Perceptron), support vector machines, k-nearest neighbours, Gaussian mixture model, naive bayes, decision tree, RBF classifiers, random forest, disciminant analysis, linear discriminant analysis, quadratic discriminant analysis, discriminant analysis—principal component analysis, partial least squares discriminant analysis, generalized distance regression and elastic net classification. The classifier parameter determined in this manner is valid for the same experimental setup in future individual tests.

It is not relevant to give an exact threshold value for the classifier parameter. A relevant threshold value can be obtained by correlating the sensitivity and specificity and the sensitivity/specificity for any threshold value. A threshold value resulting in a high sensitivity results in a lower specificity and vice versa. If one wants to increase the positive predictive value of the test to determine whether a CML patient will respond to TKI therapy or treatment, then the threshold value of the test can be changed which as a consequence will decrease the negative predictive value of the test to determine whether the CML patient will not respond to targeted treatment. If one wants to increase the negative predictive value of the test to determine whether CML patient will not respond to targeted therapy, then the threshold value can be changed in the opposite direction which as a consequence will decrease the positive predictive value of the test to determine whether CML patient will respond to targeted therapy

It is thus up to the diagnostic engineers to determine which level of positive predictive value/negative predictive value/sensitivity/specificity is desirable and how much loss in positive or negative predictive value is tolerable. The chosen threshold level could be dependent on other diagnostic parameters used in combination with the present method by the diagnostic engineers.

In yet another embodiment, the present invention relates to a method according to the present invention wherein said classifier parameter predicts the response of said patient to treatment with said TKI if said classifier parameter is above a first predetermined threshold level, and wherein said classifier parameter indicates non-response of said patient to treatment with said TKI if said classifier parameter is below a second predetermined threshold level.

According to another embodiment, the present invention relates to the method of the present invention wherein said phosphorylation level or said classifier parameter predicts a response, non-response or undetermined or intermediate prediction of the effect of said treatment with said TKI on said patient.

As used in the present application the prediction of response to treatment with TKI and preferably Imatinib, of CML patients is generally divided into two types of non-responders and responders and additionally some undetermined or intermediate responders. Whereas responders to the TKI therapy will survive longer due to the treatment, the non-responders or patients who will develop resistance to the TKI therapy will not benefit from this therapy. The method of the present invention specifically enables the distinction between responders and non-responders to the TKI therapy.

More preferably the present invention relates to a method according to the present invention wherein said phosphorylation sites are present on proteins, peptides or peptide mimetics immobilized on a solid support, and preferably a porous solid support. Preferably said immobilized kinase substrates carrying phosphorylation sites will be immobilized proteins, peptides or peptide mimetics. In a preferred embodiment of the present invention peptides are immobilized on a solid support.

As used herein “peptide” refers to a short truncated protein generally consisting of 2 to 100, preferably 2 to 30, more preferably 5 to 30 and even more preferably 13 to 18 naturally occurring or synthetic amino acids which can also be further modified including covalently linking the peptide bonds of the alpha carboxyl group of a first amino acid and the alpha amino group of a second amino acid by eliminating a molecule of water. The amino acids can be either those naturally occurring amino acids or chemically synthesized variants of such amino acids or modified forms of these amino acids which can be altered from their basic chemical structure by addition of other chemical groups which can be found to be covalently attached to them in naturally occurring compounds.

As used herein “protein” refers to a polypeptide made of amino acids arranged in a linear chain and joined together by peptide bonds between the carboxyl and amino groups of adjacent amino acid residues.

As used herein “peptide mimetics” refers to organic compounds which are structurally similar to peptides and similar to the peptide sequences list in Table 1. The peptide mimetics are typically designed from existing peptides to alter the molecules characteristics. Improved characteristics can involve, for example improved stability such as resistance to enzymatic degradation, or enhanced biological activity, improved affinity by restricted preferred conformations and ease of synthesis. Structural modifications in the peptidomimetic in comparison to a peptide, can involve backbone modifications as well as side chain modification.

For measuring the kinase activity of the sample a large variety of methods and formats are known in the art. The kinase activity can for example be measured using ELISA and multiplex ELISA techniques, blotting methods, mass spectrometry, surface plasmon resonance, capillary electrophoresis, bead arrays, macroarrays, microarrays or any other method known in the art. Depending on the type of kinase activity measurement method the solid support on which the proteins, peptides or peptide mimetics are fixed may vary. Whereas in ELISA the protein kinase substrates are attached to the surface of the microtiterplates, in microarrays the protein kinase substrates are immobilized on and/or in the microarray substrate.

In a preferred embodiment of the present invention the protein kinase substrates are immobilized on an array, and preferably a microarray of protein kinase substrates wherein the protein kinase substrates are immobilized onto a solid support or another carrier. The immobilization can be either the attachment or adherence of two or more protein kinase substrate molecules to the surface of the carrier including attachment or adherence to the inner surface of said carrier in the case of e.g. a porous or flow-through solid support.

In a preferred embodiment of the present invention, the array of protein kinase substrates is a flow-through array. The flow-through array as used herein could be made of any carrier material having oriented through-going channels as are generally known in the art, such as for example described in PCT patent publication WO 01/19517. Typically the carrier is made from a metal oxide, glass, silicon oxide or cellulose. In a particular embodiment the carrier material is made of a metal oxide selected from the group consisting of zinc oxide, zirconium oxide, tin oxide, aluminium oxide, titanium oxide and thallium; in a more particular embodiment the metal oxide consists of aluminium oxide.

Accordingly, in a further embodiment of the present invention said array is a Pamchip®.

In a further embodiment, the present invention relates to a method according to the present invention wherein said solid support (microarray) comprises any of the peptides as listed in Table 1 immobilized thereto.

In a further embodiment, the present invention relates to a method according to the present invention wherein said solid support (microarray) comprises each of the peptide as listed in Table 1 immobilized thereto.

In a further embodiment, the present invention relates to a method according to the present invention wherein said patient shows primary resistance to TKI treatment and preferably to treatment with Imatinib.

In a further embodiment, the present invention relates to a method according to the present invention wherein said patient shows secondary resistance to TKI treatment and preferably to treatment with Imatinib.

With primary resistance is meant resistance in patients that never respond to TKI treatment and preferably to treatment with Imatinib. In secondary resistance is meant resistance in patients which first respond to TKI treatment and preferably to treatment with Imatinib, but after a few months or years, resistance occurs.

In a further embodiment, the present invention relates to a method according to the present invention wherein said method predicts the resistance of said CML patient to treatment with Imatinib.

Another embodiment of the present invention regards a method for diagnosing CML in patients, wherein the kinase activity of a sample, obtained from said patient, is measured, wherein said kinase activity measurement provides phosphorylation profiles of said sample thereby diagnosing CML. Said method may further be used to determine whether said patient provides a resistance to TKI treatment.

In a further embodiment, the present invention relates to a method for diagnosing a CML patient according to the present invention wherein said phosphorylation profiles comprise the phosphorylation levels of phosphorylation sites present in at least 10 peptide markers as listed in Table 1.

Preferably also this method will use two or more of said peptide markers as described above. More preferably this method will use at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95, 100, 105, or 108 of the peptide markers listed in Table 1.

Another embodiment of the present invention regards a computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method according to the present invention.

The present invention further relates to a computer system comprising a processor, and a memory coupled to said processor and encoding one or more programs, wherein said one or more programs instruct the processor to carry out the methods according to the present invention.

The present invention also relates in another embodiment to a kit for predicting the response of a patient diagnosed with chronic myelogenous leukaemia (CML), to a tyrosine kinase inhibitor (TKI), comprising at least one array comprising immobilized proteins, peptides or peptide mimetics comprising phosphorylation sites present in at least 10 peptide markers as listed in Table 1, and optionally a computer readable storage medium having recorded thereon one or more programs for carrying out the method according to the present invention. More preferably said array comprises immobilized proteins, peptides or peptide mimetics comprising, preferably one or more, phosphorylation sites present in at least 10, 11, 12, 13, 14, 15, 16, 17, 18, 19, 20, 21, 22, 23, 24, 25, 26, 27, 28, 29, 30, 31, 32 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95, 100, 105 or 108 peptide markers as listed in Table 1.

In a preferred embodiment said proteins, peptides or peptide mimetics are at least 25% of proteins, peptides or peptide mimetics on said array. Said arrays may further comprise one or more immobilized proteins, peptides or peptide mimetics which are used as calibration means for performing the methods according to the present invention.

More particularly said array comprises immobilized proteins, peptides or peptide mimetics comprising, preferably one or more, phosphorylation sites as described in detail above representing the peptide markers as listed in table 1. Additionally said proteins, peptides or peptide mimetics are preferably at least 25%, at least 50%, at least 70%, at least 80%, at least 90% or 100% of the proteins, peptides or peptide mimetics on said array.

According to a particular embodiment the present invention pertains to a method for predicting the discontinuation or the stopping of tyrosine kinase inhibitor treatment to patients diagnosed with CML but who are unware of being cured and continue to receive tyrosine kinase inhibitor treatment, comprising the steps of:

(a) measuring the kinase activity of a sample, obtained from said patient diagnosed with CML, who continues to receive tyrosine kinase inhibitor (TKI) treatment, by contacting said sample with at least one protein kinase substrate, thereby providing a phosphorylation profile of said sample, said phosphorylation profile comprising the phosphorylation levels of phosphorylation sites present in at least 10 peptide markers as listed in Table 1; and,
(b) determining from said phosphorylation profile the disease free status of said patient.

More preferably said TKI is Imatinib.

Due to the difficult diagnostical methods for CML, often the treatment with TKI, and preferably Imatinib, is often continued even after the patient has been cured, leading to unnecessary costs and pain for the patient. The method according to the present invention therefore contributes to a faster diagnosis of cured patients and therefore overcomes the difficulties of the prior art methods.

Since the present inventors have identified a surprisingly useful set of peptide markers to be used in methods for determining the prediction of response to treatment or therapy with TKI and preferably Imatinib of a CML patient, the skilled man may carry out any method as defined above wherein he measures the kinase activity of any of the peptide markers of Table 1. Also this method may be carried out using the amount and type of peptides, proteins or protein mimetics as defined above. The formats for carrying out these methods are also as for the methods described above.

The present invention is hereafter exemplified by the illustration of particular, non-limiting examples.

### EXAMPLES

### Example 1

### Example Showing how Responders and Non-Responders to Treatment with Imatinib can be Differentiated According to a Phosphorylation Profile

Bone marrow aspirations were collected for routine-control purposes at the moment of CML diagnosis and every three months following the diagnosis. 18 Imatinib sensitive versus 19 Imatinib resistant patients were followed, of which the samples were collected 3 months to 1.5 year before resistance emergence.

For the obtained samples cytogenetic deviations in the form of BCR-Abl translocations were measured as a control. The outcomes of these measurements were used to stage the disease status of the patient and determine whether the disease was in a chronic phase (steady chronic or potentially upcoming resistance), blast phase or acute phase. Apart from the steady chronic phase, so established by constant control of the disease state through intake of Imatinib, all other stages of the disease reflecting emerging resistance against Imatinib, required immediate clinical intervention. In most cases resistance is caused by a mutation in the kinase domain of BCR-Abl thereby obstructing the inhibitory effect of the kinase inhibitor.

The samples under study consisted of a collection of thirty seven patients of which the bone marrow samples were collected on the moment of diagnosis until about 22 months after diagnosis. Apart from the patients of which the samples were taken at the moment of diagnosis. All patients were under constant Imatinib treatment at the moment the samples were collected.

The bone-marrow aspirates were layered on a Ficoll Hypaque gradient (BD) and after centrifugation the mononuclear layer was collected, spun in a pellet and the cleared pellet was stored deep-frozen in portions of 3 to 5 10⁶ cells. Frozen samples were lysed (M-Per) in an end-concentration of 5.0 10⁷ cells per milliliter under standard conditions. 5.0 10⁶ cells were used per assay and incubated on a Pamchip under standard conditions.

Collected pictures of the incubations were quantified using proprietary software (BioNavigator) and data analysis was also performed using this software in combination with the R-software package (R version 2.10.1 (2009-12-14); Copyright (C) 2009; The R Foundation for Statistical Computing; ISBN 3-900051-07-0). The (open acces) end-library enabling the testing of the wide variety of classification algorithms was CMA (“Classification for Micro Arrays”—M.Slaski et al, BMC Bioinformatics 2008, 9:439; http://www.biomedcentral.com/1471-2105/9/439).

A grid search with a number of peptide phosphorylation features ranging from no selection via 5, 10 20 40 to 80 features selected with a method selected from a list of 12 feature selection procedures was applied with 22 different classification algorithms.

A low misclassification rate of 15.4% (4 misclassifications) was found with:

a) elasticnet feature selection and the random forest or tree-based gradient boosting procedure having a standard error of 0.072, a sensitivity of 94.1% and a specificity of 66.7%.
b) “Lasso”—i.e. L1 penalized logistic regression feature selection and the random forest or tree-based gradient boosting procedure having a standard error of 0.072, a sensitivity of 94.1% and a specificity of 66.7%.

A random permutation of the class labels resulted in a misclassification rate of around 0.5 as would be expected.

The results indicate that the methods according to the present invention can be used to provide the minimal error rate when classifying new samples into responders and non-responders to treatment with Imatinib.

### Example 2

Example Showing how CML-Patients can be Differentiated from Non-CML Patients According to a Phosphorylation Profile.

The samples under study consisted of a collection of eighty-six proven CML-patients of which the blood samples were collected on the moment of diagnosis until about 22 months after diagnosis. Apart from the patients of which the samples were taken at the moment of diagnosis, all patients were under constant Imatinib treatment at the moment the samples were collected. Blood samples of twenty-two patients collected for various reasons without any known connection to CML were used as CML negative samples.

The blood samples (4 to 8 ml) were layered on a Ficoll Hypaque gradient (BD) and after centrifugation the mononuclear layers were collected, spun in a pellet and the cleared pellets were stored deep-frozen in portions of 3 to 5 10⁶ cells.

Frozen samples were lysed (M-Per) in an end-concentration of 5.0 107 cells per milliliter under standard conditions. 5.0 10⁶ cells were used per assay and incubated on a Pamchip under standard conditions.

Images collected during every kinetic step of the dynamic incubation on the Pamchip were quantified using proprietary software (BioNavigator) and data analysis was also performed using this software in combination with the R-software package (R version 2.10.1; The R Foundation for Statistical Computing; ISBN 3-900051-07-0). The (open acces) end-library enabling the testing of the wide variety of classification algorithms was CMA (“Classification for Micro Arrays”—M.Slaski et al, BMC Bioinformatics 2008, 9:439; http://www.biomedcentral.com/1471-2105/9/439).

A grid search with a number of features ranging from no selection via 5, 10, 20, 40, to 80 features selected with a method selected from a list of 12 feature selection procedures was applied with 22 different classification algorithms.

A low misclassification rate of 10% or lower was found with:

a) “t-test”-based feature selection and the “support vector machine” based classification scheme performance having a standard error of 0.01, a sensitivity of 95% and a specificity of 84%.
b) “Boosting”-based feature selection and the “k-nearest neighbour” classification scheme having a standard error of 0.01, a sensitivity of 95% and a specificity of 78%.

Other classification algorithms provided misclassification rates lower than 20%.

The results indicate that the methods according to the present invention can be used to provide the minimal error rate when differentiating CML-patients from non-CML patients.

1. A method for predicting the response of a patient diagnosed with chronic myelogenous leukaemia (CML), to a tyrosine kinase inhibitor (TKI), comprising the steps of:
(a) measuring the kinase activity of a sample, obtained from said patient diagnosed with CML, by contacting said sample with immobilized protein kinase substrates, thereby providing a phosphorylation profile of said sample, said phosphorylation profile comprising the phosphorylation levels of phosphorylation sites present in at least 10 peptide markers as listed in Table 1; and,
(b) determining from said phosphorylation profile the response of said patient to said TKI.

2. Method according to claim 1, wherein said TKI is Imatinib.

3. Method according to claim 1, wherein step (b) is replaced by calculating a classifier parameter from said phosphorylation profile; and determining the expected response of said patient to treatment with said TKI on the basis of said classifier parameter.

4. Method according to claim 3, wherein said classifier parameter predicts the response of said patient to treatment with said TKI if said classifier parameter is above a first predetermined threshold level, and wherein said classifier parameter indicates non-response of said patient to treatment with said TKI if said classifier parameter is below a second predetermined threshold level.

5. Method according to claim 1, wherein said differential phosphorylation level or said classifier parameter predicts a response, non-response or undetermined or intermediate prediction of the effect of said treatment with said TKI on said patient.

6. The method according to claim 1, wherein said phosphorylation sites are present on proteins, peptides or peptide mimetics immobilized on a solid support, and preferably a porous solid support.

7. The method according to claim 1, wherein said patient shows primary resistance to TKI treatment.

8. The method according to claim 1, wherein said patient shows secondary resistance to TKI treatment.

9. The method according to claim 1, wherein said method predicts the resistance of said CML patient to treatment with Imatinib.

10. A method for diagnosing CML for a patient, wherein the kinase activity of a sample, obtained from said patient, is measured, wherein said kinase activity measurement provides phosphorylation profiles of said sample thereby diagnosing CML for said patient.

11. The method according claim 10, wherein said phosphorylation profiles comprise the phosphorylation levels of phosphorylation sites present in at least 10 peptide markers as listed in Table 1.

12. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of claim 1.

13. A kit for predicting the response of a patient diagnosed with chronic myelogenous leukaemia (CML), to a tyrosine kinase inhibitor (TKI), comprising at least one array comprising immobilized proteins, peptides or peptide mimetics comprising phosphorylation sites present in at least 10 peptide markers as listed in Table 1, and a computer readable storage medium having recorded thereon one or more programs for carrying out the method of claim 1.

14. A method for predicting the discontinuation of tyrosine kinase inhibitor treatment to patients diagnosed with CML but who are unware of being cured and continue to receive tyrosine kinase inhibitor treatment, comprising the steps of:
(a) measuring the kinase activity of a sample, obtained from said patient diagnosed with CML, who continues to receive tyrosine kinase inhibitor (TKI) treatment, by contacting said sample with immobilized protein kinase substrates, thereby providing a phosphorylation profile of said sample, said phosphorylation profile comprising the phosphorylation levels of phosphorylation sites present in at least 10 peptide markers as listed in Table 1; and,
(b) determining from said phosphorylation profile the disease free status of said patient.

15. Method according to claim 14, wherein said TKI is Imatinib.

16. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of claim 10.

17. A kit for predicting the response of a patient diagnosed with chronic myelogenous leukaemia (CML), to a tyrosine kinase inhibitor (TKI), comprising at least one array comprising immobilized proteins, peptides or peptide mimetics comprising phosphorylation sites present in at least 10 peptide markers as listed in Table 1, and a computer readable storage medium having recorded thereon one or more programs for carrying out the method of claim 10.

18. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of claim 14.

19. A kit for predicting the response of a patient diagnosed with chronic myelogenous leukaemia (CML), to a tyrosine kinase inhibitor (TKI), comprising at least one array comprising immobilized proteins, peptides or peptide mimetics comprising phosphorylation sites present in at least 10 peptide markers as listed in Table 1, and a computer readable storage medium having recorded thereon one or more programs for carrying out the method of claim 14.
